(19) United States
(12) Patent Application Publication
(10) Pub. No.: US 2024/0428908 A1
(43) Pub. Date: Dec. 26, 2024

(54) Method and System for Converting Facsimile Documents to Electronic Health Record Formats

(71) Applicant: Consensus Cloud Solutions, LLC, Los Angeles, CA (US)

(72) Inventors: Francis Michael Toscano, III, Kendall Park, NJ (US); Menik Seneviratne, Culver City, CA (US); Jeffrey Sullivan, Rancho Palos Verdes, CA (US); Matthew Coyne Baker, Boston, MA (US); Ashley James, Crossroads, TX (US); Bronwen Patricia Huron, North Olmsted, OH (US)

(21) Appl. No.: 18/755,290

(22) Filed: Jun. 26, 2024

Related U.S. Application Data
(60) Provisional application No. 63/510,263, filed on Jun. 26, 2023.

(51) Int. Cl. G16H 10/60; G06F 16/11

(52) U.S. Cl. CPC G16H 10/60; G06F 16/116

(57) ABSTRACT

A system for preparing documents in a standards compliant electronic health record (EHR). A document intake device is configured to receive an image file transmitted to the device using an image file transport protocol. An artificial intelligence/machine learning (AI/ML) model is configured to extract patient demographic and medical information contained within the image file as a text file with the patient demographic and medical information, and format the text file for use as a EHR by a transformation engine. The transformation engine is configured to convert the text file formatted for use as the EHR into the standards compliant EHR and transmit the standards compliant EHR as a Direct Secure Message to a Health Information Service Provider (HISP). The HISP configured to send the standards compliant EHR as a Direct Secure Message for storage as a patient record if a patient match is found.

### BACKGROUND OF THE INVENTION

An electronic medical record, or EMR, was the more common name until it was determined that an electronic health record or EHR was more inclusive. Either way, these records systems primarily serve as the “source of truth” for clinicians and are where they primarily document their work and look for orders, labs, and new information on their patients. Because of this, billing and quality reporting also use this tool to measure clinical work.

In 2015 the Office of the National Coordinator, or ONC, began certifying that EHR meets specific minimum requirements for large hospitals as a part of Meaningful Use. All ONC Certified EHRs must support the delivery of documents via The Direct Standard™ (ONC FAQ Sheet) (2023 Interoperability Standards Advisory (Page 196)

Most importantly, ONC Certified EHRs are required to accept and process these files. (2023 Interoperability Standards Advisory (pg. 187).

The protocols are used because different healthcare providers, such as hospitals, clinics, and laboratories need to share medical information about patients securely and efficiently. However, each provider may use different systems and formats for storing and exchanging this information, which can make sharing and accessing patient data challenging.

Direct Secure Messaging (DSM) can utilize both XDR (Cross-Enterprise Document Reliable Interchange) and XDM (Cross-Enterprise Document Media Interchange) in conjunction with Consolidated Clinical Document Architecture (C-CDA) to facilitate the secure exchange of health information. Here is how these components work together:

### Direct Secure Messaging (DSM):

DSM is a secure messaging protocol designed specifically for healthcare providers to exchange clinical information electronically. It enables the secure transmission of patient health records, lab results, referrals, and other medical documents between authorized individuals and organizations. There are many different names for how EHRs have implemented The Direct Standard™. DSM is content agnostic, so while it can use XDR, XDM, and C-CDAs it can also use PDFs and other images. EHRs also use XDR and XDM for exchange, whereas DSM is essentially an email inbox, which does not require an EHR. (Direct Secure Messaging Basics: Q&A for Providers) (NextGen: NextGen Share Direct Messaging User Guide).

### XDR (Cross-Enterprise Document Reliable Interchange):

XDR is an Integrating the Healthcare Enterprise (IHE) profile that defines a standardized method for securely sharing healthcare documents across different organizations. DSM can use XDR as a transport mechanism within its framework. It ensures that healthcare documents, such as Continuity of Care Documents (CCDs), are packaged, transmitted, and received reliably and securely. CCD is a standard format for exchanging structured clinical information, including patient demographics, medical history, medications, allergies, etc.

### XDM (Cross-Enterprise Document Media Interchange):

XDM is another IHE profile focusing on exchanging medical media files, such as images and accompanying documents. While DSM primarily handles text-based messages, it can securely leverage XDM to exchange media files. This allows healthcare providers to send and receive medical images, reports, and related documents using the same secure messaging infrastructure provided by DSM. These documents need to be sent as a zipped file. A zipped file is one created using a well known compression technique.

### C-CDAs (Consolidated Clinical Document Architecture):

C-CDAs are a standardized format for representing clinical documents in a structured manner. They are based on the HL7 Clinical Document Architecture (CDA) standard. C-CDAs provide a way to exchange patient health information in a standardized and interoperable format. Within the DSM framework, C-CDAs can be securely transmitted using XDR, ensuring that the clinical documents are packaged and delivered reliably to authorized recipients. (Consolidated CDA Overview|HealthIT.gov) (Internal Education Presentation)

FIG. 1 shows a prior art system in which a health care provider sends a document by fax 7 or non-fax 9 via a transport protocol 11 which is dependent on the format of the document such as a fax protocol (e.g., TIFF) or a pdf protocol (e.g., % PDF-1.4). Upon receipt by an intake process, the document is manually reviewed and the data on the received document is manually entered 13 creating, for example, a C-CDA document. The manually created C-CDA document is then provided by DSM to a Health Information Service Provider (HISP) 15 which delivers by DSM the standardized healthcare document to a system 17 which has EHR with patient matching enabled. When a patient match is found, the data from the C-CDA document is automatically inserted into a patient record 19a. If a patient match is not found, the C-CDA is document is placed into a review queue 19b for manual review so that a patient record can be located or created if necessary.

Although the prior art system shown in FIG. 1 is highly automated, the critical step of creating the C-CDA document or equivalent is a mostly manual step which is expensive, time consuming and error prone.

### SUMMARY OF THE INVENTION

Original hard copies of health record documents become data in an Electronic Health Record (EHR) format with the use of a fax number or other document identifier, Artificial Intelligence (AI)/Machine Learning (ML), Transformation Engine and a Secure Delivery Mechanism.

1. The process starts with an unstructured image usually captured as a fax or a scanned image which is then processed as described below.

2. Using advanced AI and ML, demographic labels and values are identified such as a “patient first name” to create an identified label. Other labels may be identified based on this list which include by way of example only:
a. First Name
b. Last Name
C. Middle Name (including middle initial)
d. Name Suffix
e. Date of Birth
f. Date of Death
g. Sex
h. Address
i. Phone Number
j. Phone Number Type
k. Email Address

USCDI Patient Demographics/Information available from www.healthit.gov is used to help guide which fields are extracted.

3. A transformation engine then converts the extracted data from the AI into a structured and predictable format based on the Consolidated Clinical Document Architecture (C-CDA), which is the most widely used format for health information exchange in the US today.

4. From this, a Continuity of Care Document (CCD) is created. A CCD template gives a snapshot of a patient's health record in C-CDA format. The CCD is delivered via Direct/XDR/XDM to a Health Information Services Provider (HISP) for delivery to any 2015 Certified EHR as is well known in the art. Since the Direct Transport protocol has a size limit of 25 Megabytes, any files that exceed this limit are identified, and the user is notified to review the document within a HIPAA compliant specialized portal, where they can review and download any data needed.

5. A Health Information Service Provider, or HISP, is an accredited network service operator that enables nationwide clinical data exchange using Direct Secure Messaging (aka Direct, Direct Messaging and the Direct Project). Direct is a widely used healthcare protocol for secure, verified data transmission, that is similar to email, with Direct Addresses as unique identifiers. HISPs and Direct are regulated and monitored by the DirectTrust.org, a governance organization empowered by HHS. Using the Direct Secure Address provided by the customer, what started as an unstructured secure image (fax or scan) is securely routed, from which pertinent information is extracted and transformed into a Continuity of Care Document, to be easily ingested by a 2015 certified EHR.

### BRIEF DESCRIPTION OF THE DRAWINGS

FIG. 1 is a block diagram showing the elements and process flow of a prior art system for transferring Electronic Medical Records.

FIG. 2 is a block diagram showing the elements and process flow of a system for transferring Electronic Medical Records using artificial intelligence to replace manual review and entry.

FIG. 3 is a block diagram showing the process flow of the artificial intelligence/machine language (AI/ML) model and transformation elements of the invention.

### DETAILED DESCRIPTION OF THE INVENTION

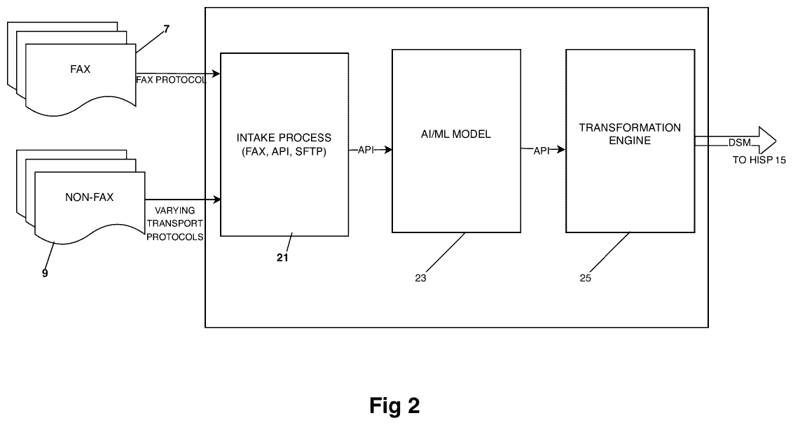

FIG. 2 shows the process flow for how an image containing medical data is analyzed via artificial intelligence to create a CCD with a C-CDA structure. The CCD with a C-CDA structure is then securely routed via a HISP to a certified EHR.

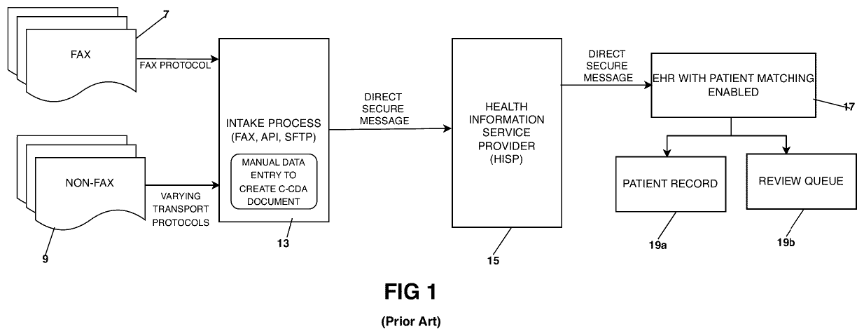

As shown in FIG. 2, a faxed 7 or scanned 9 image is received 21 and then passed by an Application Program Interface (API) or equivalent to an artificial intelligence/machine language (AI/ML) system 23 for processing. The AI/ML system analyzes the data on the received image and extracts patient data from the image. If the image contains data for more than one person, the patient data for each person is separated to correspond to the data for one person. The patient data is then sent by an API or equivalent and checked for validity and formatted by a transformation engine 25 into a CCD with a C-CDA structure. If for some reason the patient data is not validated, the user who provided the image is notified (not shown in FIG. 2). Otherwise, the validated data is securely routed via DSM or equivalent to a HISP 15 and then to a certified EHR 17 for further handing as described above with reference to FIG. 1.

As should be apparent, blocks 21, 23 and 25 of FIG. 2 generally correspond to block 13 of FIG. 1.

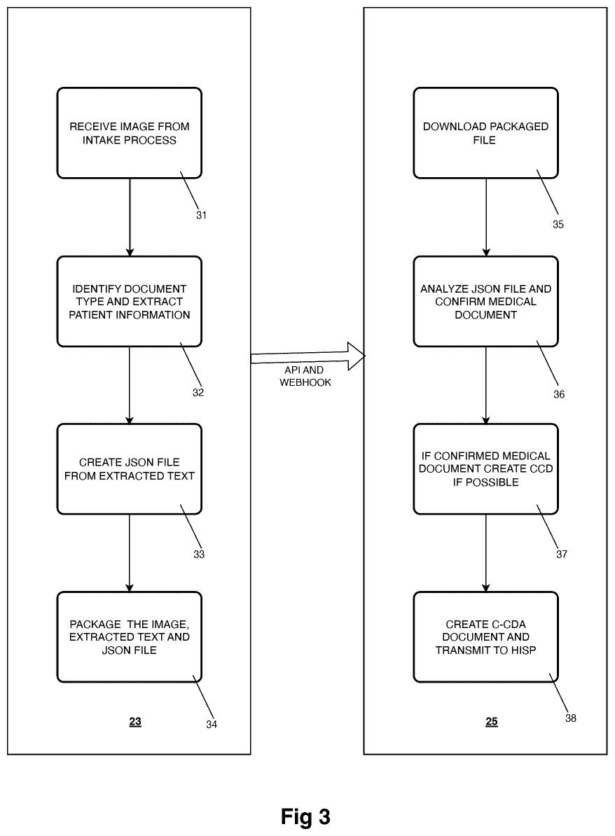

FIG. 3 provides further details for blocks 23 and 25 of FIG. 2.

Referring now to FIG. 3, AI/ML model 23 is an AI system which takes as its input an image file such as TIFF (e.g., from a fax) or pdf (e.g., from an email) (with embedded text and extracts the text into a text file. If an image contains content for multiple patients, the content relevant to each patient is separated so that separate CCD's can be created for each patient.

AI systems which can extract text from image files are well known such as imagetotext.io which is a website which uploads an image file, extracts the text and generates the extracted text in a new text file. Newocr.com and nanonets.com are similar. Although optical character recognition (OCR) systems extract text from image files, AI variants can extract text from complex images with text within tables or other complications. While an initial step by an AI/ML model may be to extract the text from an image by OCR, the AI/ML model then uses its artificial intelligence to create a more accurate result than is possible with OCR alone, especially for complex images. While the AI/ML model is trained to perform this specific task, the techniques used to perform such training are not specific to the invention and are well known in the art, and, therefore, not detailed herein.

The present invention provides a text extraction capability which provides an output which can be fed directly into transformation engine 25. The AI/ML model is trained to isolate images that contain content that is not medical in nature. The user will be notified of these images and will be provided access to a specialized portal (not shown), where the user can review and download any data or take any other action as needed as to content which is not medical in nature.

Specifically, AI/ML model 23 receives 31 a fax or scanned image. Once the image is obtained, as part of the intake process, the text is extracted by OCR and data correction techniques are applied such as spell and grammar check adapted for use with medical records which can have atypical spelling and grammar. The cleaned up document is further processed 32 using AI as follows.

The clean document includes fields such as reason for referral, patient demographic information and provider information. The document is first confirmed to be a medical document by checking for certain key details depending on the type of the document (i.e., referral, x-ray, intake form, etc.) and identified as the type of document based on such key details. Patient demographics along with specified questions related to the document types are pulled from the created text file. If necessary, the document is separated by patient or document type in order to facilitate individual transactions to the EHR to trigger the workflow. For example, if the document type is referral, information such as the date of service and ICD-10 code (10th revision of the International Statistical Classification of Diseases and Related Health Problems) is pulled in order to trigger and facilitate the workflow within the EHR. It should be noted that the workflow can be configured by the customer with questions modified by the customer to support their workflow. That is, although this description provides a relatively generic description of the necessary workflow, any modifications which may be necessary for specific situations are easily implemented by one skilled in the art.

A JavaScript Object Notation (JSON) formatted document is created 33 from this data by the AI/ML model 23. Of course, any standardized format for storing and transporting data can be used instead of JSON. Techniques for generating JSON formatted documents from text files are also well known in the art. For example, the invention creates a JSON from the extracted text which is the raw text obtained from OCR. The AI parses the text and organizes the information into a structured JSON format:
“document type”: “; ab report”: type of document being processed
“patient first name”: “stacy”: Represents the patients first name.
“patient last name”: “jones”: Represents the patients last name
“patient gender”: female: Represents the gender of the patient
“patient date of birth”: 05/04/1969e: Represents the patients DOB
“patient gender”: female: Represents the gender of the patient
“patient phone number”: 732 555 1212: Represents the phone number
“patient address”: 123 tall ave, pines, NJ 08824: Represents the address (may be split up by road, city, state and zip as needed)
“patient email address”: sto@gmail.com: Represents the email address
“Is this document a medical form, ‘Yes’ or ‘No’?” Determines communication behavior for Spam filtering

JSON (JavaScript Object Notation) is used because it's a lightweight data interchange format that's easy for machines to parse and generate. Each piece of information from the extracted text corresponds to a key-value pair in the JSON object.

This JSON object can be easily used by other programs or AI systems for further processing, such as database storage, information retrieval, or integration into other applications.

The AI uses natural language processing (NLP) techniques to identify and extract specific pieces of information from unstructured text, and then convert that structured data into JSON or another suitable format for processing or storage. Depending on the document type, e.g., a referral, additional questions may be asked pertaining to the document, such as date of service and ICD-10 code in order to facilitate the EHR workflow.

The AI is trained to provide a confidence score based for example on the number of errors it found to correct and upper and lower ranges of the converted data and any other third party input.

The 1) original fax or PDF image (which can be searchable PDF) is packaged with 2) the text file of the extracted text (handwritten and typed fonts), and 3) the JSON file which can include the confidence score, range validations, third party query validations, National Provider Identifier (NPI) database and positioning coordinates. The package is a zip file containing the three specified documents.

The zip file is then received 35 by transformation engine 25. The steps needed to convert the JSON file to a C-CDA document are as follows. The received zip file is downloaded and the JSON file is used to and perform the following functions:

For each package (identified by a file naming convention), the package is decompressed and the JSON file is analyzed to confirm it is a medical document. This is done by checking that the JSON file includes certain key terms that are unique to medical documents such as patient demographics as described above.

If the document is not a medical document (e.g., spam fax), an alternate email address provided by the customer is notified to inform the customer where they can then look at the fax manually in a portal. This prevents fax spam from being sent to the EHR.

If yes, defined fields are located (regardless of the document type, the map is the same either the data exists or it does not). If there is not enough data to create the CCD (certain fields are required like name, etc.) then the file without the CCD is attached.

In an embodiment, other CDA document types can be created but most EHRs are not fully mapped to support them.

This creates 38 a file in C-CDA format and which is then securely transmitted to HISP 15 for the subsequent processing as explained in FIG. 1. The manner in which the file in C-CDA format is created is as follows.

The AI provides a zip file that contains the JSON, the original PDF or fax image and the OCR text file which is delivered to a document creation system, via, for example, Webhook. Once the zip file is downloaded and decompressed, a mapping engine consumes the JSON file and creates a CDA using a template that has been pre built. The JSON file content is mapped to the CCD and constructed to comply with CMS standards.

The CCD and PDF image is then packaged as an SMPTS message and delivered via a HISP to the direct address.

As an example, an original source document will normally have spaces for items such as Reason for Referral, Patient, Name, Date of Birth, Birth Sex, Race, Preferred Language, Current Address, and Phone Number; Provider Name and address and contact information. The specifics of how this data is extracted is dependent on the AI/ML model which is used with one example being as described above with reference to FIG. 3.

Assuming the text of the extracted data is as follows:
Reason for referral: Pulmonary Function Tests
Patient Name: John Smith
Patient Date of Birth: Dec. 1, 1960
Patient Birth Sex: Male
Patient Race: Caucasian
Patient Preferred Language: English
Patient Address: 22 Jones Street, Albany, NY
Patient Phone: 555-555 5555
Provider Name: Dr Edward Johnson
Provider Office Address: 57 Adams Street, Albany, NY
AI/ML model 23 will output a JSON file such as:

```
{
″Reason for referral: Pulmonary Function Tests
“Patient”: {
  “Name”: {
    “data” : “John Smith”,
    “bounding_box” : {
      “top_left_x” : “200”,
      “top_left y” : “900”,
      “bottom_left_x” : “400”,
      “bottom_left y” : “1100”.
    }
  }
  “Date of Birth”: {
    “data” : “12/01/1960”,
    “bounding_box” : {
      “top_left_x” : “300”,
      “top_left y” : “1000”,
      “bottom_left_x” : “500”
      “bottom_left y” : “1200”.
    }
  }
  “Birth Sex”: {
    “data” : “male”,
    “bounding_box” : {
      “top_left_x” : “400”,
      “top_left y” : “1100”,
      “bottom_left_x” : “600”,
      “bottom_left y” : “1300”,
    }
  }
  “Race”: {
    “data” : “caucasian”,
    “bounding_box” : {
      “top_left_x” : “600”,
      “top_left y” : “1400”,
      “bottom_left_x” : “700”,
      “bottom_left y” : “1400”
    }
  }
  “Preferred Language”: {
    “data” : “English”,
    “bounding_box” : {
      “top_left_x” : “600”
      “top_left y” : “1400”,
      “bottom_left_x” : “800”.
      “bottom_left y” : “1500”,
    }
  }
  “Address”: {
    “data” : “22 Jones Street, Albany, NY”,
    “bounding_box” : {
      “top_left_x” : “600”,
      “top_left y” : “1400”,
      “bottom_left_x” : “800”,
      “bottom_left y” : “1500”
    }
  }
  “Phone”: {
    “data” : “555-555-5555”,
    ”bounding_box” : {
      “top_left_x” : “600”,
      “top_left y” : “1400”,
      “bottom_left_x” : “900”.
      “bottom_left y” : “1600”,
    }
  }
  “Address”: {
    “data” : “22 Jones Street, Albany, NY”,
    “bounding_box” : {
      “top_left_x” : “600”,
      “top_left y” : “1400”,
      “bottom_left_x” : “800”,
      “bottom_left y” : “1500”,
    }
  }
“Provider”: {
  “Name”: {
    “data” : “Dr Edward Johnson”,
    “bounding_box” : {
      “top_left_x” : “600”,
      “top_left y” : “1400”,
      “bottom_left_x” : “900”,
      “bottom_left y” : “1600”,
    }
  }
  “Address”: {
    “data” : “57 Adams Street, Albany, NY”,
    “bounding_box” : {
      “top_left_x” : “600”,
      “top_left y” : “1400”,
      “bottom_left_x” : “1000”,
      “bottom_left y” : “1700”
    }
  }
}
```

This JSON file is then input to transformation engine 25 which takes the JSON file output by AI/ML model 23 and generates an output file which is formatted as a Healthcare standard accepted by all 2015 certified EHR's such as a C-CDA as explained above with reference to FIG. 3.

A C-CDA document created from this JSON file would be:
Reason for Referral
Pulmonary Function Tests
Patient Demographics
Name: John Smith
Date of Birth: Dec. 1, 1960
Birth Sex: Male
Race: Caucasian
Patient Preferred Language: English
Address: 22 Jones Street, Albany, NY
Phone: 555-555 5555
Provider name and contact information
Provider's Name: Dr Edward Johnson
Provider's office contact information: 57 Adams Street, Albany, NY

Transformation engine 25 operates on the JSON file to create the C-CDA document is explained above with reference to FIG. 3 or via an Application Program Interface (API) as one example. The techniques needed to create such an API are well known to persons skilled in the art, the specifics of which are apparent from the description above with reference to FIG. 3.

The C-CDA document plus the original from transformation engine 25 are delivered as a Direct Secure Message or equivalent to a Health Information Service Provider (HISP) 15 which delivers via DSM, or equivalent, the standardized healthcare document to an EHR with patient matching enabled 17 as described above with reference to FIG. 1.

An embodiment of the invention may be implemented as an article of manufacture in which a non-transitory machine-readable storage medium has stored thereon instructions which program one or more data processing components (generically referred to here as “a processor”) to perform the operations described above. For example, in one embodiment, the above-described functions described with reference to blocks 31-38 of FIG. 3, respectively may be performed by a processor or processors that is execute instructions stored in the non-transitory machine-readable storage medium. The non-transitory machine-readable storage medium may be a part of the AI/ML model 23 and transformation engine 25, as described herein. In other embodiments, some of these operations might be performed by specific hardware components that contain hardwired logic. Those operations might alternatively be performed by any combination of programmed data processing components and fixed hardwired circuit components.

While certain embodiments have been described and shown in the accompanying drawings, it is to be understood that such embodiments are merely illustrative of and not restrictive on the broad disclosure, and that the disclosure is not limited to the specific constructions and arrangements shown and described, since various other modifications may occur to those of ordinary skill in the art.

We claim:

1. A system for preparing documents in a standards compliant electronic health record (EHR) comprising:
a document intake device configured to receive an image file transmitted to the device using an image file transport protocol;
an artificial intelligence/machine learning (AI/ML) model configured to extract patient demographic and medical information contained within said image file as a text file with said patient demographic and medical information, and format said text file for use as said EHR by a transformation engine;
said transformation engine configured to convert said text file formatted for use as said EHR into said standards compliant EHR and transmit said standards compliant EHR as a Direct Secure Message to a Health Information Service Provider (HISP);
said HISP configured to send said standards compliant EHR as a Direct Secure Message for storage as a patient record if a patient match is found.

2. The system defined by claim 1 wherein the AI/ML model is configured to:
receive a fax or scanned image with embedded text information and extract said text information by optical character recognition and apply data correction techniques including spell and grammar check adapted for use with medical records to obtain a clean document including fields with patient demographic information and provider information;
create a document which includes said fields in a standardized format for storing and transporting data;
package the received fax or scanned image with the extracted text information and said document which includes said fields in said standardized format into a compressed file.

3. The system defined by claim 1 where said transformation engine is configured to:
receive a compressed file containing fields including patient demographic and medical information, decompress the received file to obtain a document which includes said fields in a standardized format and confirm that said document is a medical record;
locate predetermined defined fields in said decompressed file and create a Continuity of Care Document (CCD) with said defined fields as a Consolidated Clinical Document Architecture (C-CDA) document;
transmit the C-CDA document to a Health Information Service Provider (HISP.

4. The system defined by claim 3 wherein said standardized format for storing and transporting data is a JavaScript Object Notation (JSON) formatted document.

5. The system defined by claim 1 wherein the AI/ML model uses natural language processing (NLP) techniques to identify and extract specific pieces of information from unstructured text, and then convert that structured data into a JSON format for processing or storage.

6. The system defined by claim 4 wherein the image file is packaged with the text file, and the JSON file as a zip file and sent to the transformation engine which decompresses the zip file and the JSON file is analyzed to confirm it is a medical document.

7. The system defined by claim 6 wherein pre-defined fields in the JSON file are located, the CCD is created and securely transmitted to the HISP.

8. The system defined by claim 7 wherein the CCD is created using a pre-built template.
